(19) United States
(12) Patent Application Publication
(10) Pub. No.: US 2007/0067036 A1
(43) Pub. Date: Mar. 22, 2007

(54) Hydrogel total disc prosthesis

(75) Inventors: Robert Garryl Hudgins, Burnsville, MN (US); Michael E. Lancial, St. Louis Park, MN (US)

Correspondence Address: WOOD, HERRON & EVANS (ZIMMER SPINE), 2700 CAREW TOWER, 441 VINE STREET, CINCINNATI, OH, 45202, US

(73) Assignee: Zimmer Spine, Inc., Minneapolis, MN (US)

(21) Appl. No.: 11/230,870

(22) Filed: Sep. 20, 2005

(51) Int. Cl. A61F 2/44

(52) U.S. Cl. 623/17.13; 623/17.12

(57) ABSTRACT

A prosthetic spinal disc includes a cushioning member encased in a flexible sac and positioned between a pair of opposing endplates. A pair of retainer members are also located inside the sac to secure the sac to the endplates. The cushioning member is expandable from a first configuration to a second configuration. The disc is insertable into the spinal column as a single piece with the cushioning member in the first configuration and thereafter expanded to the second configuration to support and space apart adjacent vertebrae. The cushioning member may be replaced or revised without disturbing the placement of the endplates.

### TECHNICAL FIELD

The present invention is related to spinal stabilization devices. More particularly, this invention relates to a prosthetic disc for replacing a natural disc and a method of implanting a prosthetic vertebral disc.

### BACKGROUND

The spinal column is a highly complex system of bones and connective tissues that provides support for the body and protects the delicate spinal cord and nerves. The spinal column includes a series of vertebrae stacked one on top of the other, each vertebral body including an inner or central portion of relatively weak cancellous bone and an outer portion of relatively strong cortical bone. Situated between each vertebral body is an intervertebral disc that cushions and dampens forces experienced by the spinal column. A vertebral canal containing the spinal cord and nerves is located behind the vertebral bodies.

There are many types of spinal column disorders including scoliosis (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of one vertebra over another, usually in a lumbar or cervical spine) and other disorders caused by abnormalities, disease, or trauma, such as ruptured or slipped discs, degenerative disc disease, fractured vertebra, and the like. Patients that suffer from such conditions usually experience extreme and debilitating pain, as well as diminished nerve function. Such disorders can also limit range of movement and threaten the critical elements of the nervous system housed within the spinal column.

One well known method of treating these spinal disorders via surgical intervention is to immobilize regions of the spine, usually by fusing or joining adjacent vertebrae to one another. A variety of systems have been disclosed in the art which achieve this immobilization by implanting artificial assemblies in or on the spinal column. These assemblies may be classified as anterior, posterior, or lateral implants. As the classifications suggest, lateral and anterior assemblies are coupled to the anterior portion of the spine, which is the sequence of vertebral bodies. Similarly, posterior implants are located on the posterior portion of the spine and generally comprise pairs of rods, which are aligned along the axis which the bones are to be disposed, and hooks coupled to the lamina or to the transverse processes or screws inserted through the pedicles.

Surgical interventions also include full or partial disc replacement. These methods are considered flexible spinal stabilization methods as they do not result in complete spinal fusion. Full disc replacement involves removal of existing disc material and implantation of a prosthetic insert. The prosthetic insert usually includes a cushioning core coupled to endplates which are in turn fixed to adjacent spinal regions. Clinical successes of artificial discs varies. It has been difficult to provide a prosthetic disc approximating both the cushioning and flexibility of a natural spinal disc. Furthermore, typical replacement discs have a multi-part construction requiring a lengthy, complicated multi-step implantation procedure. Finally, artificial discs are usually difficult to revise following implantation.

What is needed then is a total disc replacement that approximates the cushioning and flexibility of a natural spinal disc and has a simple implantation and revision procedure.

### SUMMARY

In one embodiment, the present invention is a prosthetic spinal disc for insertion into a space between vertebrae. The disc prosthesis includes a pair of endplates that may be attached to adjacent vertebrae and a cushioning member positioned in between the endplates and surrounded by a flexible sac. The cushioning member may be made of hydrogel material which expands upon hydration or an in situ curable elastomer which is injected into the sac after the disc prosthesis is implanted. The sac may be made of a textile having a three-dimensional honeycomb weave which can be woven to limit the amount of torsion and radial expansion the disc prosthesis permits. The sac is secured to the endplates to hold the disc together. The disc may further include a pair of retainer members that are attachable to the endplates to help secure the sac to the endplates. The entire disc prosthesis may be inserted into a space between adjacent vertebrae while the cushioning member is in a collapsed configuration. After insertion, the cushioning member is expanded to space apart the endplates so that the disc prosthesis support the adjacent vertebrae in a spaced relationship.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 illustrates a side view of a human spinal column including a prosthetic disc.

FIG. 2 illustrates a prosthetic disc according to one embodiment of the present invention that includes a cushioning core sandwiched between opposing endplates.

FIG. 3A illustrates a bottom view of an endplate according to one embodiment of the present invention.

FIG. 3B illustrates a perspective view of the endplate of FIG. 3A.

FIG. 4 illustrates a cross-sectional view of the prosthetic disc of FIG. 2 taken along line 4-4.

FIG. 5A illustrates a perspective view of a retainer member according to one embodiment of the present invention.

FIG. 5B illustrates a side view of the retainer member of FIG. 5A.

FIG. 5C illustrates a top view of the retainer member of FIG. 5A.

FIG. 6 illustrates a sac having an open sleeve configuration according to one embodiment of the present invention.

FIG. 7A illustrates a prosthetic disc according to another embodiment of the present invention that includes a slot in the endplates for lateral insertion of the retainer members.

FIG. 7B illustrates a top view of the endplate of FIG. 7A.

FIG. 7C illustrates a perspective view of the retainer member of FIG. 7A.

FIG. 8 illustrates a perspective view of a retainer member and a bifurcated endplate.

### DETAILED DESCRIPTION

The present invention pertains to a modular total disc prosthesis. A total disc prosthesis in accordance with the present invention is surgically revisable, allows a less invasive approach for most lumbar spine levels, and an anterolateral approach for the L5-S1 disc space.

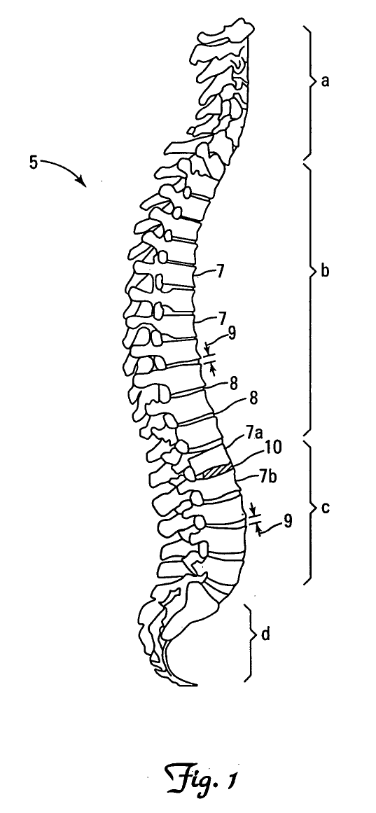

FIG. 1 illustrates a human spinal column 5 including vertebrae 7 belonging to one of a cervical region a, a thoracic region b, a lumbar region c and a sacral region d of the spinal column 5. Intervertebral discs 8 are positioned in intervertebral spaces 9 between adjacent vertebrae 7. One of the discs 8 of the lumbar region c has been partially removed and replaced with a prosthetic spinal disc 10. Disc 10 is secured to adjacent vertebrae 7a and 7b and provides cushioning and flexion in a manner simulating natural discs 8. Disc 10 also serves to space apart the adjacent vertebrae 7a and 7b a distance similar to that provided by a natural disc. While prosthetic spinal disc 10 is shown implanted into the lumbar region c, prosthetic spinal disc 10 is easily adapted in size and shape for implantation into the cervical region a and thoracic region b.

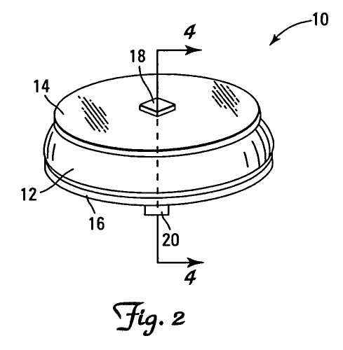

FIG. 2 illustrates the prosthetic spinal disc 10 according to one embodiment of the present invention. The disc 10 includes a cushioning core 12 sandwiched between a pair of opposing endplates 14, 16. The cushioning core 12 forms a spacer member spacing apart the endplates 14, 16, and thus the vertebrae to which the endplates 14, 16 are attached. The cushioning core 12 is engageable with the endplates 14, 16 to attach the endplates 14, 16 to one another to form the prosthetic disc 10.

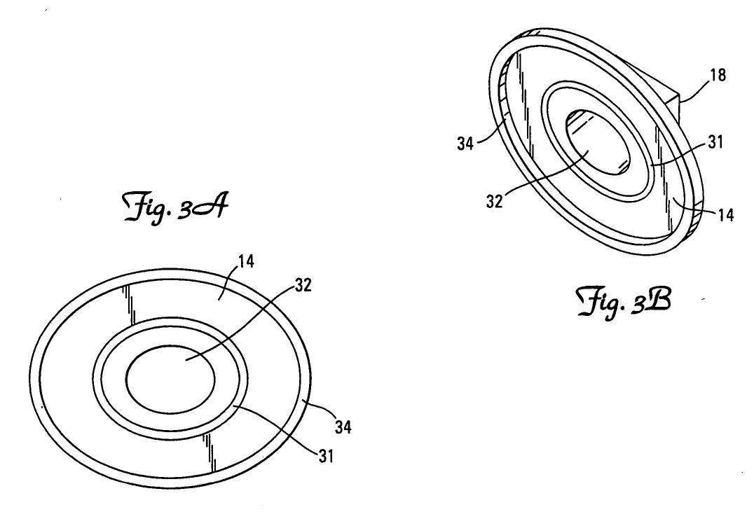

FIGS. 3A and 3B illustrate one of the endplates 14, 16 in greater detail. In one embodiment, the endplates 14, 16 have a shape that corresponds to the shape of the vertebrae 7, such as a generally elliptical shape. However, other shapes may also be used, for example, circular, oval, etc.

In one embodiment, endplates 14, 16 include centrally located keels 18, 20, respectively, as anchoring members for affixing to upper and lower vertebrae 7a and 7b at implantation to fix the disc 10 into place. The keels 18, 20 may take on a variety of configurations to facilitate fixation according to a variety of means. In one embodiment, the keels 18, 20 may be shaped (e.g., square) to reduce rotation of the disc 10 relative to the vertebrae 7. The keels 18, 20 may also include other means for fixing the disc 10 within the spine, including outer threading, locking pin arrangements, etc.

According to one embodiment, at least an outer surface 40 of the endplates 14, 16 is formed of, or coated with, a porous material chosen to permit or promote bone and/or tissue ingrowth. An exemplary porous material is described in U.S. Pat. No. 5,282,861 to Kaplan, which is hereby incorporated herein by reference. Such materials also include porous metals, for example, open cell tantalums, including Trabecular Metal™ (available from Zimmer Inc. at www.zimmer.com). Such materials, along with or in place of the keels 18, 20 and other fixation structures previously described, provide a means for fixing the disc 10 within the spine.

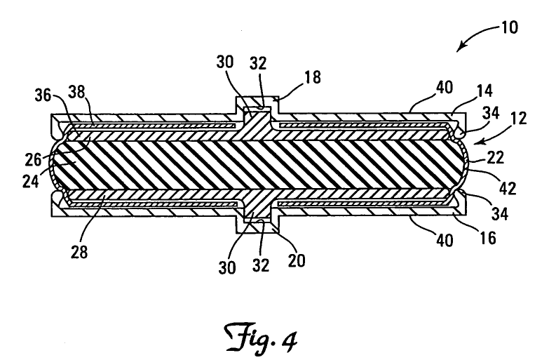

FIG. 4 illustrates the interior arrangement of the spinal disc 10 of FIG. 2. As shown in FIG. 4, the cushioning core 12 includes a flexible sac 22 encasing or surrounding a cushioning member 24 sandwiched between opposing retainer members 26, 28. The retainer members 26, 28 are attached to the respective endplates 14, 16. The friction therebetween holds the sac 22 in place, forming the disc 10. The retainer members 26, 28 may be generally flat, disc or plate shaped, and may have a similar shape as the endplates 14, 16. The endplates 14, 16 may also include a circumferential lip 34 or other means to reduce migration of the sac 22 and retainer members 26, 28 relative to the endplates 14, 16.

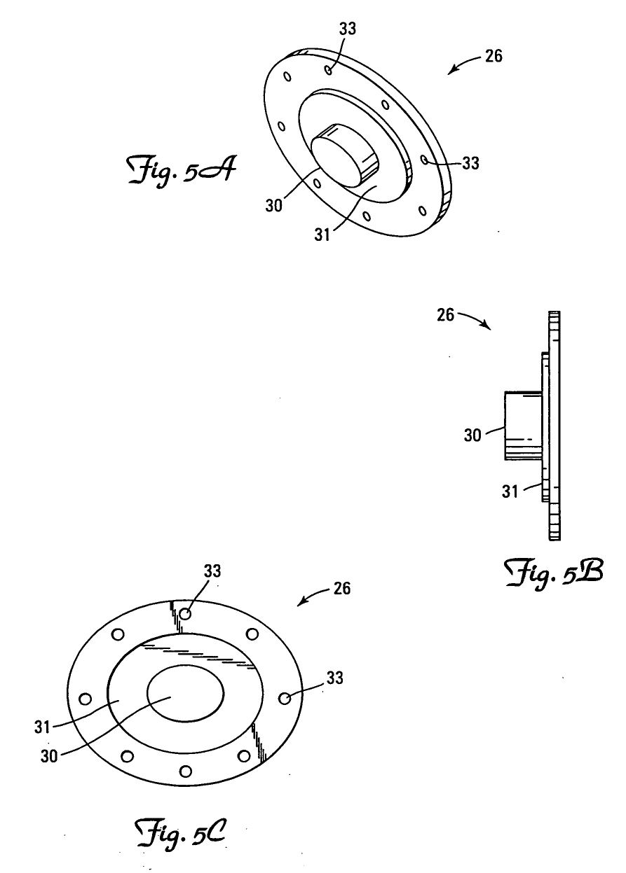

The retainer members 26, 28 and endplates 14, 16 may include a locking mechanism to facilitate securing the retainer members 26, 28 to the endplates 14, 16 to hold the sac 22 in a stable position. In the embodiment shown in FIGS. 4 and 5A-5C, the retainer members 26, 28 each include a boss 30 shaped to cooperate with a recess 32 extending into the keels 18, 20 in mating engagement. The retainer members 26, 28 may include a series of bosses, as illustrated by secondary boss 31 in FIGS. 5A-5C, to provide a stronger boss structure. In one embodiment, the boss 30 and recess 32 have elliptical or other non-circular shapes to prevent rotation of the retainer members 26, 28 with respect to the endplates 14, 16. The locking mechanism may include other locking means, including, for example, a snap fit mechanism or complementary threading.

The endplates 14, 16 are formed of a substantially rigid material chosen to withstand stress and friction from the adjacent vertebrae 7a, 7b. In one embodiment, the retainer members 26, 28 are somewhat more flexible than the endplates 14,16. The retainer members 26, 28 are further formed with a smooth, low friction outer surface 36 and have an outer vertical profile with a full radius shaped to reduce wear on an adjacent inner surface 38 of the sac 22. A surface is “low-friction” as used herein when the material and other characteristics, such as morphology, are appropriately chosen for the object in contact with the surface such that, under normal physiological conditions over the expected lifetime of the implant, the surface has a sufficiently low coefficient of friction such that it is intended to not produce in either the object or the surface a degree of wear that renders the implant unusable over the expected lifetime of the implant.

The endplates 14, 16 and retainer members 26, 28 may be formed of any of several types of materials that are biocompatible or bio-inert and have good strength and rigidity/flexibility characteristics. One such type of material is a biocompatible engineering polymer, such as polyetheretherketone (PEEK), polyaryletherketone (PAEK), polyimide, polysulfone, fiber forms of polyethylene terephthalate (PET) (also known as Dacron®), polyetherimides and liquid crystalline polymers. Another such type of material is a thermoplastic engineering elastomer, such as polyurethane or any other engineering elastomer having suitable stiffness and wear properties. Yet another such type of material is a biocompatible metal, such as titanium or stainless steel.

The endplates 14, 16 and retainer members 26, 28 may be formed of various combinations of the above-described materials and need not be formed of the same grade of material, nor even the same type of material. Different materials or grades of materials may also be used to provide different flexibility and surface textures between the endplates and the retainer members 26, 28. For example, porous or open cell materials need not be employed for the retainer members 26, 28, as the retainer members 26, 28 may not be exposed to tissues.

The cushioning core 12 is expandable from a first, collapsed configuration to a second, expanded configuration. In the first configuration, the cushioning core 12 has a lower profile, which allows the endplates 14, 16 to have a reduced separation therebetween. In this collapsed configuration, the prosthetic spinal disc 10 may be inserted between adjacent vertebrae that are compressed or at least which have not been fully distracted. This allows for a less invasive surgical insertion. In the second configuration, the cushioning core 12 supports the endplates 14, 16 a further distance apart from one another. The cushioning core 12 may be sized such that in the second configuration the prosthetic disc 10 supports or distracts the adjacent vertebrae apart from one another by a chosen distance. In one embodiment, the disc 10 is sized and shaped to have a vertical displacement of from about 5 mm to about 20 mm from the first configuration to the second configuration. In one embodiment, the cushioning core 12 provides increased cushioning in the second configuration relative to the first configuration.

The cushioning member 24 may be formed of any suitable material known in the art of intervertebral disc replacement. In one embodiment, this includes hydrogel polyurethanes, polyacrylamide, polyvinyl alcohol (PVA), and any other hydrogel material having suitable fluid content, swelling pressure, elastic modulus (linear or non-linear), biocompatibility, wear properties and biodurability. The cushioning member 24 may be a single mass of material, as is shown in the accompanying figures, or may consist of multiple pieces or pellets of material.

In one embodiment, the cushioning member 24 is dehydrated in the first configuration and hydrated in the second configuration. When dehydrated, the cushioning member 24 is substantially smaller than when in a fully hydrated state. This permits the retainer members 26, 28 to collapse towards one another, reducing the overall height of the disc 10, and facilitating insertion of the disc 10 between closely spaced vertebrae 7. However, when hydrated, as is shown in FIGS. 1 and 2, the hydrogel material of the cushioning member 24 includes a substantial liquid component. Following hydration, the cushioning member 24 expands or swells to substantially inflate or expand the sac 22 and takes on a greater cushioning or shock absorbing quality. The cushioning member 24 may be hydrated after implantation by irrigating a lateral surface 42 of the sac 22 with a fluid such as saline. The irrigation liquid permeates the sac 22 and is absorbed by the cushioning member 24. Bodily fluids permeating the sac 22 are absorbed by the cushioning member 24 in addition to, or in place of, irrigation.

Use of the term “dehydrated” is not meant to infer or require that the cushioning member 24 had a liquid or a fluid content which was subsequently removed. By “dehydrated” it is merely meant that the cushioning member 24 substantially lacks a liquid or fluid content. Furthermore, the terms “hydrated” and “dehydrated” merely refer to the absence or presence of liquid or fluid content, and are not intended to refer specifically to water content. The cushioning core 24 may be formed of material such as lyogel which is “hydrated” with a fluid other than water. In other words, the cushioning member 24 may be expanded or swollen from a first configuration to a second configuration upon the addition of non-water based fluids such as oil-based fluids.

In one embodiment, the cushioning member 24 may be formed of an elastomer material having suitable elastic modulus (linear or non-linear), biocompatibility, wear properties and biodurability. Suitable elastomers include polyurethanes, styrene-butadiene-rubbers and silicone rubbers. Preferably, such elastomer materials are curable in situ. The disc 10 may be inserted into an intervertebral space in a collapsed configuration in which the elastomer material of the cushioning member 24 has not yet been installed. Following insertion of the disc 10 into the disc space, the elastomer material of the cushioning member 24 is injected into the sac 22, expanding the sac 22. After the elastomer material has cured, the cushioning member 24 has load bearing capabilities. The cushioning member 24 may also be formed of an in situ curable elastomer having a hydrogel component, as is described in U.S. Pat. No. 6,443,988, which is herein incorporated by reference.

The sac 22 may be formed of a variety of materials, including ultra high molecular weight, highly oriented, highly crystalline, drawn or spun polyethylene fibers, such as Spectra® 1000 (available from Honeywell International, Inc. of Morristown, N.J.) and Dyneema® (available from DSM Dyneema of Geleen, the Netherlands). Other suitable sac materials include polyaramides such as Kevlar® 49 and Kevlar® 149 (available from DuPont of Richmond, Va.), and polyethylene terephthalate (PET).

In one embodiment, the sac 22 is provided with a coating chosen to give the sac 22 a smoother lateral surface 42 and to reduce leakage of the cushioning member 24 (not shown). The coating may be formed of elastomeric or hydrogel material.

In one embodiment, the sac 22 is formed of a textile material. Textile fabrication processes for making the sac 22 include two- and three-dimensional variations of braiding, knitting and weaving. One exemplary three-dimensional weave pattern is known as a honeycomb weave. A honeycomb weave has a three-dimensional, cell-like structure in which long floats form the periphery of each cell. The interlacing is progressively tightened, towards the cell centers, with the tightest interlacing occuring at the center of the cell. This weave pattern creates a structure of hollow pickets between raised portions, similar to a waffle. The face and back of such a woven textile or fabric are similar, with the midpoint on the cell on one side serving as the outer corner on the other side. In other words, the high point on one side of the textile or fabric is the low point on the other side. An exemplary honeycomb weave pattern is available at Offray Speciality Narrow Fabrics, Inc., of Chester, N.J.

A honeycomb weave provides a cell structure having a substantially completely interconnected porosity in three dimensions throughout the fabric. Such porosity facilitates fluid transfer into and out of the hydrogel material of the cushioning member 24 encased in the sac 22. For example, fluids may transfer into and out of the cushioning member 24 through the fabric of the sac 24 while the sac 24 restrains extrusion and/or migration of the material of the cushioning member 24 through the fabric. The porous structure also facilitates additional processing that may be performed on the cushioning member 24, such as the application of an elastomeric or hydrogel coating through the sac 22. A porous or open construction to the sac 22 may also be provided according to other means of braiding, weaving and knitting and are also contemplated by the present invention. Furthermore, even if the cushioning member 24 is formed of an elastomer type material rather than a hydrogel-type material, the sac 22 may permit the transfer of bodily fluids into and out of the sac 24.

In one embodiment, the sac 22 is formed of a woven fiber that has been woven in such a manner so as to resist radial expansion of the cushioning member 24 when subject to longitudinal compressive forces. To accomplishing this aim, fibers of the sac 22 may be generally vertically oriented. In one embodiment, the fibers of the sac 22 are preferably oriented from about 60° to about 90° from a vertical axis. In this manner, the construction of the textile of the sac 22 generally permits vertical extension and flexion and resists radial expansion and torquing.

In one embodiment, the sac 22 is sized such that when the cushioning core 12 is in the first or collapsed configuration the sac 22 is flaccid and when the cushioning core 12 is in the second, or expanded, configuration, the sac 22 is substantially taut. Thus, when the disc 10 is thereafter subjected to vertical loading, the cushioning member 24 expands radially against the sac 22, inducing a tensile load on the sac 22 resisting any radial expansion. This tensile load helps to support the spinal column 5 when undergoing vertical load components, as when standing or moving while upright.

In one embodiment, the cushioning member 24 is a means for absorbing energy and functions as a load bearing shock absorber and spacer between the endplates 14, 16 in much the same manner as a natural intervertebral disc 8. The cushioning member 24 also permits asymmetric stretching and compression, as well as asymmetric shear movement, i.e., the movement of the cushioning member 24 in differing directions in a generally horizontal plane. Finally, the cushioning member 24 is capable of torquing or torsional movement. The endplates 14, 16 are thus able to move and tilt towards and away from one another about a 360° circumference, allowing the user to stretch, flex and extend the spinal column 5 in a natural manner. Thus, in another embodiment, the cushioning core 12 is a means of allowing spinal movement (i.e., movement of one endplate relative to the other) in a number of degrees of freedom.

The combination of the ability of the cushioning member 24 to compress, flex and shear asymetrically and the constraint against radial expansion and torquing provided by the sac 22 provides the prosthetic spinal disc 10 with the ability to replicate the loci of instantaneous centers of rotation of the healthy, natural motion segment. The cushioning member 24 and sac 22 also provide damping and shock absorbing capabilities to help protect the surrounding spinal column, particularly the adjacent vertebra 7a and 7b (See FIG. 1). Finally, because the retainer members 26 and 28 are adapted to reduce frictional wear and tear on the sac 22, the prosthetic spinal disc 10 has a lower wear particle production rate. This is also facilitated by the relatively small motions of individual components relative to one another.

Various combinations of materials for the endplates 14, 16, retainer members 26, 28 and sac 22 materials may provide improved longevity of the disc 10. In one embodiment, the endplates 14, 16 and retainer members 26, 28 are made of stainless steel or titanium and the sac 22 is made of Spectra® 1000. In another embodiment, the endplates 14, 16 and retainer members 26, 28 are made of engineering polymers or harder grades of thermoplastic engineering elastomers and the sac 22 is made of a polyaramid such as Kevlar® 49 or Kevlar® 149. In another embodiment, the endplates 14, 16 and retainer members 26, 28 are made of thermoplastic engineering elastomers and the sac 22 is made of PET.

The end plates 14, 16, retainer members 26, 28 and sac 22 are shown secured to one another frictionally or mechanically via the locking mechanism as illustrated in FIGS. 2 and 4. Accordingly, the cushioning core 12 may be released from the endplates 14, 16 to facilitate revision or replacement of the prosthetic disc 10. In other embodiments, the sac 22 is thermally or chemically bonded to either or both of the endplates 14, 16 and retainer members 26, 28 alone or in combination with the locking mechanism. For example, FIGS. 5A-5C show bond regions 33 on the retainer members 26, 28 for bonding to the sac 22 and/or the endplates 14, 16. Chemical bonding is accomplished by forming chemically active regions on adjacent surfaces of components and bonding the surfaces together. In addition, the sac 22 may be adhered to either or both of the endplates 14, 16 and retainer members 26, 28 with an adhesive. Suitable adhesives are biocompatible or bioinert, particularly with respect to any adhesive outside of the sac 22, as would be any adhesive bonding the sac 22 to the endplates 14, 16. Other methods well known in the art can be used to affix the various components to one another.

In one embodiment, shown in FIG. 4, the sac 22 is woven around the retainer members 26, 28, substantially encasing the retainer members 26, 28. Weaving a fabric around an object is well known in the art of textile fabrication. In another embodiment, the sac 22 is formed integrally with the retainer members 26, 28 through a molding process. In this version, the sac 22 and retainer members 26, 28 are formed of compatible materials. Combinations of compatible materials include Kevlar, polyaramid fiber and PET sac 22 materials with a PEEK or a rigid polyurethane retainer member 26, 28.

In another embodiment, all or a portion of the prosthetic spinal disc 10 is formed of a material adapted to prevent the adhesion of tissue, for example, scar tissue, to the prosthetic spinal disc 10. Such materials include hyaluronic acid, a material know to discourage the formation of scar tissue. Such a material may form a coating over all or a portion of the prosthetic spinal disc 10. In one embodiment, the endplates 14, 16 and sac 22 are coated with such a material. By reducing the volume of scar tissue tending to form around and adhere to the prosthetic spinal disc 10, the prosthetic spinal disc 10 is more easily revised post-implantation.

Prior to implantation of disc 10 in surgery, vertebral disc material in the disc space 9 into which the prosthetic disc 10 is to be implanted is removed as necessary. Recesses are formed, as appropriate, in the vertebral bodies 7a and 7b above and below the disc space 9 for receiving the keels 18, 20, respectively. Disc 10 is fully assembled prior to insertion with the cushioning member 24 dehydrated such that the sac 22 is partially collapsed and the disc 10 has a reduced height. The vertebrae 7a and 7b can be distracted if necessary. The disc 10 is then inserted in a single step in the gap 9 left by the removed disc 8 between adjacent vertebrae 7a and 7b. The keels 18, 20 are positioned in the respective recesses in the adjacent vertebrae 7a and 7b to secure the disc 10 in position. The cushioning member 24 is then hydrated. Hydration may be accomplished by transfer of bodily fluids through the sac 22 or by irrigation using other fluids, such as water or a saline solution. Alternately, when the cushioning member 24 is formed of an elastomer material, the prosthetic disc 10 is inserted as described above after which the elastomer material is injected into the sac 22 to expand the sac 22. After the elastomer material is injected, the elastomer material is allowed to cure, if necessary, to attain the desired consistency and load bearing capabilities.

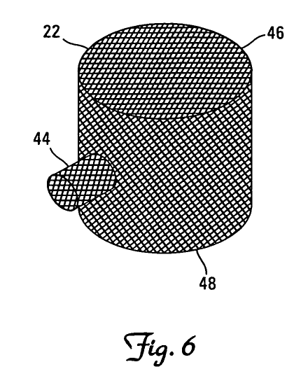

In another embodiment, as is shown in FIG. 6, the sac 22 is shaped like tubular sleeve and includes a channel for providing access to the cushioning member 24 for direct irrigation or injection and as a conduit for insertion or removal of all or portions of the cushioning member 24. In one embodiment, as is shown in FIG. 6, the channel is a secondary sleeve 44 woven from the sac 22. The ends 46, 48 of the sac 22 are pulled around the retainer members 26, 28 such that upon securing of the retainer members 26, 28 to the endplates 14, 16, the sac 22 is also secured. The sac 22 is drawn tightly around the retaining plate 26, 28 using a draw string (not shown) or secured with adhesive or by thermal processes. In this manner, the cushioning member 24 may be inserted into the sac 22 after the sac 22 has been implanted along with the retainer members 26, 28 and endplates 14, 16. Should the cushioning member 24 require replacement or revision, it may be accessed without compromising the weave structure of the sac 22.

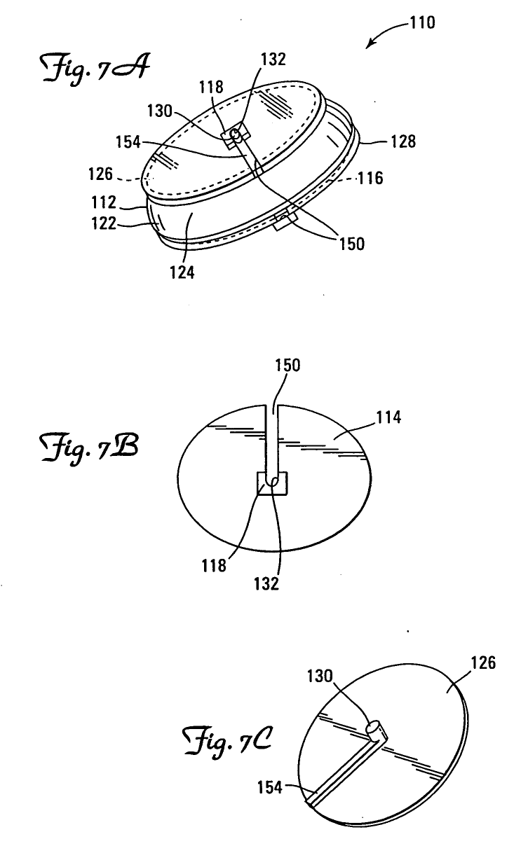

FIGS. 7A-7C show a prosthetic spinal disc 110 in another embodiment of the present invention. The disc 110 is generally similar to the disc 10 shown in FIGS. 2 and 4, and includes a cushioning core 112 sandwiched between a pair of endplates 114, 116, and a woven sac 122 encasing a pair of retainer members 126, 128 and a cushioning member 124. However, the disc 110 is adapted for a two-step insertion procedure. The endplates 114, 116 each include a radial slot 150. The slots 150 accept the retainer member bosses 130 and guide them into mating cooperation with the inner keel recesses 132 in the endplate keels 118. The slot 150 may further include a guide groove or other feature to direct the orientation of the retainer members 126, 128 with respect to the endplates 114, 116. A radial ridge 154 extends from the boss 130 to the perimeter of the retainer member 126,128 and is sized to be received in the slot 150. The ridge 154 cooperates with the slot 150 to provide a smooth outer profile on the endplates 114, 116 when the disc 110 is fully assembled. In this manner, the endplates 114, 116 may be first positioned in the spine and fixed into place. Then, the cushioning core 112, including the cushioning member 124, retainer members 126, 128 and sac 122 are slid into place. This arrangement further allows the cushioning core 112 to be released from the endplates 114, 116 and removed or revised without disturbing the placement of the endplates 114,116.

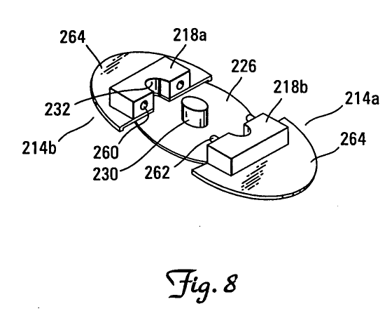

FIG. 8 shows an endplate arrangement in another embodiment in which an endplate 214 is bifurcated into opposing halves 214a and 214b. Cooperating male and female portions 260, 262 are included with the endplate halves 214a, 214b to facilitate securement to one another. The male and female portions 260, 262 may be integrated into half keels 218a 218b, as is shown in FIG. 8, or may be integrated into a plate-portion 264 of the endplate 214. The retainer member 224 is held in place with an interference or press fit between the endplate halves 214a, 214b. Alternately, the retainer member 224 is held in place with set screw or bosses that interact with the endplate halves 214a, 214b. The endplate halves 214a, 214b may be removed or revised independently of one another, and provide greater flexibility in carrying out implantation. The endplates halves 214a, 214b may also be separated to facilitate release of the cushioning core 212.

Various modifications and additions may be made to the exemplary structures and steps discussed without departing from the scope of the present invention. Various combinations, permutations, and rearrangements of those structures and steps may similarly be made without departing from the scope of the present invention. Accordingly, the scope of the present invention is intended to embrace all such alternatives, modifications, permutations and variations as fall within the scope of the claims, together with all equivalents thereof.

1. A prosthetic spinal disc for insertion into a space between vertebrae, the prosthetic spinal disc comprising:
a pair of spaced-apart endplates adapted to couple to a respective vertebra;
a cushioning member disposed between the endplates; and
a flexible sac surrounding the cushioning member and attached to the endplates.

2. The prosthetic spinal disc of claim 1 wherein the sac is attached to the endplates by being frictionally engaged to the endplates.

3. The prosthetic spinal disc of claim 1 wherein the sac is attached to the endplates by being adhered to the endplates.

4. The prosthetic spinal disc of claim 1 wherein the sac is releasably attached to the endplates.

5. The prosthetic spinal disc of claim 1 wherein the sac further comprises at least a first retainer member engageable with a first of the endplates.

6. The prosthetic disc of claim 5, wherein the at least one retainer member includes a boss and the first endplate includes a boss receiving recess.

7. The prosthetic disc of claim 5, wherein the at least one retainer member has a low friction surface adjacent an inner surface of the sac.

8. The prosthetic disc of claim 5, wherein the first endplate is bifurcated.

9. The prosthetic spinal disc of claim 1, wherein the endplates comprise a material adapted to promote tissue ingrowth.

10. The prosthetic spinal disc of claim 1 wherein at least a portion of the prosthetic spinal disc is formed of a material adapted to discourage formation of scar tissue.

11. The prosthetic spinal disc of claim 1 the cushioning member is expandable from a first configuration in which the prosthetic spinal disc is sized to be inserted into a compressed intervertebral space to a second configuration in which the prosthetic spinal disc is sized to support the vertebrae in a normal, spaced relationship.

12. A prosthetic spinal disc for insertion into a space between vertebrae, the prosthetic spinal disc comprising:
a pair of spaced-apart endplates adapted to couple to a respective vertebra; and
a spacer member disposed between the endplates, wherein the spacer member is expandable from a first configuration to a second configuration in which the spacer member is substantially cushioning; and
a flexible sac surrounding the spacer member and attached to the endplates.

13. The prosthetic spinal disc of claim 12 wherein in the first configuration the sac substantially flaccid and in the second configuration the sac is substantially taut.

14. The prosthetic spinal disc of claim 12, wherein the sac is formed from a material comprising an ultra high molecular weight, highly oriented, highly crystalline drawn or spun polyethylene, a polyaramide or a polyethylene.

15. The prosthetic spinal disc of claim 12, wherein the sac comprises a fabric having a three-dimensional weave.

16. The prosthetic spinal disc of claim 12, wherein the sac is fluid permeable.

17. The prosthetic disc of claim 12, wherein the spacer member comprises a hydrogel material.

18. The prosthetic spinal disc of claim 12, wherein the spacer member comprises one of a hydrogel polyurethane, a polyacrylamide or a polyvinyl acetate.

19. The prosthetic spinal disc of claim 12, wherein the spacer member comprises an elastomer material.

20. The prosthetic spinal disc of claim 19 wherein the elastomer material is curable in situ.

21. The prosthetic disc of claim 12, wherein the spacer member comprises one of a polyurethane, a silicone rubber or a styrene-butadiene-rubber.

22. A method of implanting a prosthetic spinal disc, the method comprising the steps of:
inserting a prosthetic spinal disc in a first configuration into space between adjacent vertebrae;
fixing the prosthetic spinal disc to the adjacent vertebrae; and
expanding the prosthetic spinal disc to a cushioning configuration in which the adjacent vertebrae are spaced apart from one another by a desired distance.

23. The method of claim 22, wherein the expanding step comprises irrigating a dehydrated hydrogel core located in the prosthetic spinal disc.

24. The method of claim 22 wherein the expanding step comprises injecting an elastomer material into the prosthetic spinal disc.

25. The method of claim 24 further comprising curing the elastomer material.

26. The method of claim 22, wherein the inserting step comprises inserting a pair of opposing endplates into the space and then inserting an expandable, cushioning member between the endplates.

27. The method of claim 22, wherein the endplates are bifurcated and wherein the inserting step further includes assembling the endplates around the cushioning member.

28. The method of claim 22 further comprising revising the prosthetic spinal disc after implantation.
